(19) United States
(12) Patent Application Publication
(10) Pub. No.: US 2021/0223251 A1
(43) Pub. Date: Jul. 22, 2021

(54) IMMUNE RESPONSE PROFILING OF TUMOR-DERIVED EXOSOMES FOR CANCER DIAGNOSIS

(71) Applicant: AGENCY FOR SCIENCE, TECHNOLOGY AND RESEARCH, Singapore (SG)

(72) Inventors: Jackie Y. Ying, Singapore (SG); Yiran Zheng, Singapore (SG); Yi Fei Lee, Singapore (SG)

(21) Appl. No.: 16/761,827

(22) PCT Filed: Dec. 6, 2018
(86) PCT No.: PCT/SG2018/050594

§ 371 Date: May 6, 2020

(30) Foreign Application Priority Data
Dec. 6, 2017 (SG) 10201710131X

(51) Int. Cl. G01N 33/574

(52) U.S. Cl. CPC G01N 33/57492

(57) ABSTRACT

Disclosed herein are methods of (i) detecting cancer or cancer type in a subject or (ii) simultaneously testing for, or distinguishing between, multiple types of cancer in a subject; methods of screening subjects for a prevalence of cancer type or cancer types; methods of managing a subject with a cancer type; and methods of identifying whether a subject having a cancer type is responding to management of that cancer type; and methods of generating a response profile specific for a cancer type. Disclosed herein also include tumor-derived exosome-induced immune response or cancer-specific response profile created based on the measurement of functional impacts of tumor-derived exosomes on immune cells in vitro for use or when used for detecting or diagnosing cancer or cancer type in a subject; tumor-derived exosome-induced immune response for use in or when used for creating a cancer-specific response profile measuring functional impacts of tumor-derived exosomes on immune cells in vitro; and use of a tumor-derived exosome-induced immune response for generating a cancer-specific response profile measuring functional impacts of tumor-derived exosomes on immune cells in vitro. Disclosed herein also include tests, assays, kits, apparatus or devises for use or when used for the method, the response, the profile or the use as disclosed herein.

### RELATED APPLICATION

This application claims the benefit of Singaporean Provisional Application No. 10201710131X filed 6 Dec. 2017, the content of which is incorporated herein by reference in its entirety.

### TECHNICAL FIELD

This invention relates, inter alia, to a method of detecting cancer or cancer type in a subject, said method comprising the step of measuring functional impacts of tumor-derived exosomes on immune cells in vitro to create a cancer-specific response profile, wherein the cancer-specific response profile is indicative of the cancer or the cancer type in the subject. The invention also relates to methods of cancer management as well as to tests, assays and kits for use in detecting or monitoring cancer or cancer type.

### BACKGROUND ART

Liquid biopsies hold great promise to cancer diagnosis as they are less invasive and allow early detection and therapy monitoring in comparison to conventional tissue biopsy. Circulating tumor cells (CTC), circulating tumor DNA/RNA and tumor-derived exosomes (TEXs) are the three tumor signatures in the blood^{1, 2, 3}. Among them, circulating tumor DNA (ctDNA) is studied most extensively. However, ctDNA might not represent the actual living tumor cells as they are released from dead or dying tumor cells, and are prone to degradation in the blood^{4, 5}. On the other hand, the applications of CTCs are limited by their scarce number, heterogeneity and methodological discrepancies^{6, 7}.

TEXs are extracellular vesicles which contain or are associated with cell-specific biomolecules, such as proteins, RNA or DNA. These biomolecules, released from actual living tumor cells, are protected by lipid bilayers and can be used as cancer biomarkers and therapeutics^{8, 9, 10, 11.} After Skog, et al. showed that RNA extracted from TEXs in patient's blood could be used to diagnose glioblastoma¹², Byron, et al. developed the first commercially available exosomal RNA-based lung cancer diagnostic kit to detect EML4-ALK mutations¹³. MicroRNA in TEXs also has served as biomarkers for ovarian cancer¹⁴. Proteins in TEXs have also demonstrated success in diagnosing colorectal cancer¹⁵ and pancreatic cancer¹⁶, etc.

To the present inventors' best knowledge, so far, all exosome-based cancer diagnosis technologies rely on content profiling, which means they look for cancer type-specific biomarkers. However, searching for biomarkers in a sea of noise from healthy cells still remains one of the biggest challenges. In addition, tumors are immensely heterogeneous at the DNA, RNA and epigenetic levels. Patient-specific interactions between cancer cells and the immune system in tumor microenvironment further increased variations from evolutionary and mutational aspects^{17, 18, 19}. Consequently, identifying a set of protein or nucleic acid biomarkers that are highly sensitive and specific to a type of cancer is technically challenging and costly. That is why despite the emergence of successful stories of cancer liquid biopsy, there is still only quite limited number of validated cancer biomarkers available to a few cancer types in clinical setting^{20, 21, 22, 23}. Furthermore, the list of biomarkers for different types of cancer is not exhaustive. Potential cancer patients might need to be diagnosed against hundreds of biomarkers to be free from a specific list of cancers. This might increase the diagnosis costs and involve the withdrawing of excessive amount of patient blood. Thus, a more broad-spectrum cancer diagnostic test that can detect multiple types of cancers simultaneously and does not rely on cancer biomarkers is needed.

Not only do TEXs contain biomarkers indicative of their parental cancer cells' identity, they also possess functional messenger molecules deployed by tumor cells to influence other cells, especially those in the immune system^{9, 24, 25, 26}. TEXs have been demonstrated to be immunosuppressive. They contain or express various combinations of immunoregulatory molecules such as IL-10, TGF-β, PD-1, PDL-1, TRAIL, FasL, CD39 and CD73 to suppress the function of T-cells, impair T-cells responses to stimulants, promote expansion of regulatory T-cells, or induce apoptosis of cytotoxic T-cells. In the meantime, TEXs can also be immunostimulatory due to their concentrated tumor antigens and heat shock proteins^{27, 25, 26, 28, 29, 30, 31, 32}.

### DETAILED DESCRIPTION OF THE INVENTION

The present inventors have now discovered that heterogeneous functional impacts of TEXs on immune cells might serve as another identity signature in addition to the cancer-specific genetic and protein information in TEXs.

According to a first embodiment of the present invention, there is provided a method of detecting cancer or cancer type in a subject, said method comprising the step of using a cancer-specific response profile, created based on the measurement of functional impacts of tumor-derived exosomes on immune cells in vitro, to identify the cancer or the cancer type in the subject, wherein said tumor-derived exosomes are isolated from the subject.

According to a second embodiment of the present invention, there is provided a method of simultaneously testing for, or distinguishing between, multiple types of cancer in a subject, said method comprising the step of using a cancer-specific response profile, created based on the measurement of functional impacts of tumor-derived exosomes on immune cells in vitro, to identify the cancer or the cancer types in the subject, wherein said tumor-derived exosomes are isolated from the subject.

According to a third embodiment of the present invention, there is provided a method of screening subjects for a prevalence of cancer type or cancer types, said method comprising the step of using a cancer-specific response profile created for each subject, created based on the measurement of functional impacts of tumor-derived exosomes on immune cells in vitro, to identify the cancer or the cancer types in each said subject, wherein said tumor-derived exosomes are isolated from the subjects.

According to a fourth embodiment of the present invention, there is provided a method of managing a subject with a cancer type, said method comprising the steps of:

(1) using a cancer-specific response profile, created based on the measurement of functional impacts of tumor-derived exosomes on immune cells in vitro, to identify the cancer type in the subject, wherein said tumor-derived exosomes are isolated from the subject; and

(2) managing the subject if the subject has been found to have the cancer type.

According to a fifth embodiment of the present invention, there is provided a method of identifying whether a subject having a cancer type is responding to management of that cancer type, said method comprising the steps of:

(1) using a cancer-specific response profile, created based on the measurement of functional impacts of tumor-derived exosomes on immune cells in vitro, to identify the cancer type in the subject, wherein said tumor-derived exosomes are isolated from the subject; and

(2) comparing the respective cancer-specific response profile created before and during and/or after management of the cancer type, wherein a change in the cancer-specific response profile identifies the subject as having responded to the management of the cancer type.

According to a sixth embodiment of the present invention, there is provided a tumor-derived exosome-induced immune response or cancer-specific response profile created based on the measurement of functional impacts of tumor-derived exosomes on immune cells in vitro for use or when used for detecting or diagnosing cancer or cancer type in a subject.

According to a seventh embodiment of the present invention, there is provided a tumor-derived exosome-induced immune response for use in or when used for creating a cancer-specific response profile measuring functional impacts of tumor-derived exosomes on immune cells in vitro.

According to an eighth embodiment of the present invention, there is provided use of a tumor-derived exosome-induced immune response for generating a cancer-specific response profile measuring functional impacts of tumor-derived exosomes on immune cells in vitro, wherein said tumor-derived exosome is isolated from a subject having cancer and said cancer-specific response profile is indicative of the cancer type in the subject.

According to a ninth embodiment of the present invention, there is provided a method of generating a response profile specific for a cancer type, said method comprising the steps of:

(1) measuring functional impacts of tumor-derived exosomes on immune cells in vitro, wherein said tumor-derived exosomes are isolated from a subject having a specific cancer type; and

(2) creating a cancer-specific response profile based on the functional impact specific for the cancer type.

According to a tenth embodiment of the present invention, there is provided a test, assay, kit, apparatus or device for use or when used for detecting or diagnosing cancer or cancer type in a subject, as described in one or more other embodiments.

According to an eleventh embodiment of the present invention, there is provided a test, assay, kit, apparatus or device for use or when used for simultaneously testing for or distinguishing between multiple types of cancer in a subject, as described in one or more other embodiments.

According to a twelfth embodiment of the present invention, there is provided a test, assay, kit, apparatus or device for use or when used for detecting or measuring a tumor-derived exosome-induced immune response, as described in one or more other embodiments.

According to a thirteenth embodiment of the present invention, there is provided a mathematical algorithm or algorithms for use or when used for measuring or quantifying at least one tumor-derived exosome-induced immune response specific for a cancer type and/or for creating a cancer-specific response profile based on the immune response specific for the cancer type, as described in one or more other embodiments.

According to a fourteenth embodiment of the present invention, there is provided a prebuilt database of reference cancer-specific response profiles for use or when used for identifying a cancer type in a subject or distinguishing between multiple types of cancer in a subject.

The subject can be a human or a different type of mammal, including: a farm animal such as a pig, cow, horse, sheep or goat; a companion animal such as a dog or cat; or, a laboratory animal such as a rabbit, mouse or rat.

Any suitable type or types of immune cells can be used. For example, the immune cells can comprise one or more of T-cells, natural killer (NK cells), and B cells. Preferably, the immune cells are T-cells. Any suitable type or types of T-cells can be used. Examples of suitable T cells include CD8 T-cells and CD4 T-cells. Particularly preferred immune cells include naïve CD8⁺ T-cells, naïve CD4⁺ T-cells, activated (Act) CD8⁺ T-cells and Act CD4⁺ T-cells.

T-cells can be sourced from any suitable organ, including mouse spleen or human peripheral blood mononuclear cells (PBMC), for example.

The method can comprise the step of measuring functional impacts of tumor-derived exosomes on immune cells in vitro to create the cancer-specific response profile and/or reference cancer-specific response profiles. This can be achieved in any suitable way.

Creating a cancer-specific response profile/the functional impacts can comprise measuring one or more of the following: suppression of the function of immune cells; impairment of immune cell responses to stimulants; promotion of expansion of regulatory immune cells; induction of apoptosis of cytotoxic immune cells; or immunostimulation.

Creating a cancer-specific response profile/the functional impacts can comprise measuring one or more of the following: suppression of the function of T-cells; impairment of T-cell responses to stimulants; promotion of expansion of regulatory T-cells; induction of apoptosis of cytotoxic T-cells; or immunostimulation.

Creating a cancer-specific response profile/the functional impacts can comprise measuring immunosuppression due to one or more of the following immunoregulatory molecules: IL-10, TGF-β, PD-1, PDL-1, TRAIL, FasL, CD39 and CD73.

Creating a cancer-specific response profile/the functional impacts can comprise measuring immunostimulatory effect due to one or more of the following molecules: tumor antigens and heat shock proteins.

Preferred examples of immunoregulatory molecules (immunosuppressive or immunostimulatory) include IL-10, TGF-β, PD-1, PDL-1, TRAIL, FasL, CD69, CD25, pSTAT5, CD39, CD73, ki67, Tim3, Granzyme B, IFNγ, CTLA4, tumor antigens and heat shock proteins as well as those described in references 27, 25, 26, 28, 29, 30, 31, 32, each of which is incorporated herein in its entirety by way of cross-reference.

Creating a cancer-specific response profile/the functional impacts can comprise measuring at least one expression level of a marker on and/or in an immune cell. Any suitable type of immune cell surface marker and/or intracellular marker or markers can be used. For example, any suitable type of T-cell surface marker or markers, and/or intracellular marker or markers can be used.

The marker can be, for example, an immune cell activation marker, an immune cell proliferation marker, an immune cell exhaustion marker, an immune cell cytotoxicity marker, an immune cell cytotoxicity and apoptosis marker, or an immune cell inhibitory marker.

In some embodiments, the activation marker can be CD69, CD25 or pSTAT5.

In some embodiments, the proliferation marker can be ki67.

In some embodiments, the exhaustion marker can be Tim3.

In some embodiments, the cytotoxicity marker can be Granzyme B or IFNγ.

In some embodiments, the cytotoxicity and apoptosis marker can be FasL.

In some embodiments, the inhibitory marker can be PD-1 or CTLA4.

Preferably, more than one type of immune marker is measured at the protein level in order to create a cancer-specific response profile.

The cancer can be any suitable type of cancer. For example, the cancer can be renal carcinoma, colorectal carcinoma (colon cancer and/or rectal cancer), skin cancer (including basal cell carcinoma, cell carcinoma, squamous cell carcinoma and melanoma), leukemia, lymphoma, tumors of the central nervous system, breast cancer, prostate cancer, cervical cancer, uterine cancer, lung cancer, ovarian cancer, testicular cancer, thyroid cancer, astrocytoma, glioma, pancreatic cancer, mesotheliomas, gastric cancer, liver cancer, renal cancer including nephroblastoma, bladder cancer, oesophageal cancer, cancer of the larynx, cancer of the parotid, cancer of the biliary tract, endometrial cancer, adenocarcinomas, small cell carcinomas, neuroblastomas, adrenocortical carcinomas, epithelial carcinomas, desmoid tumors, desmoplastic small round cell tumors, endocrine tumors, Ewing sarcoma family tumors, germ cell tumors, hepatoblastomas, hepatocellular carcinomas, non-rhabdomyosarcome soft tissue sarcomas, osteosarcomas, peripheral primitive neuroectodermal tumors, retinoblastomas, and rhabdomyosarcomas.

The method can comprise the step of comparing the created cancer-specific response profile of the subject with one or more previously created reference cancer-specific response profiles, wherein each said reference profile was created based on a subject diagnosed with a particular type of cancer.

In some embodiments, the method can comprise comparing the created cancer-specific response profile of the subject with a prebuilt database of reference cancer-specific response profiles, wherein matching or near matching subject and reference profiles indicate the type of cancer that the subject has. For example, the prebuilt reference profile database can have at least one reference profile for one or more of the following types of cancers: renal carcinoma, colorectal carcinoma (colon cancer and/or rectal cancer), skin cancer (including basal cell carcinoma, cell carcinoma, squamous cell carcinoma and melanoma), leukemia, lymphoma, tumors of the central nervous system, breast cancer, prostate cancer, cervical cancer, uterine cancer, lung cancer, ovarian cancer, testicular cancer, thyroid cancer, astrocytoma, glioma, pancreatic cancer, mesotheliomas, gastric cancer, liver cancer, renal cancer including nephroblastoma, bladder cancer, oesophageal cancer, cancer of the larynx, cancer of the parotid, cancer of the biliary tract, endometrial cancer, adenocarcinomas, small cell carcinomas, neuroblastomas, adrenocortical carcinomas, epithelial carcinomas, desmoid tumors, desmoplastic small round cell tumors, endocrine tumors, Ewing sarcoma family tumors, germ cell tumors, hepatoblastomas, hepatocellular carcinomas, non-rhabdomyosarcome soft tissue sarcomas, osteosarcomas, peripheral primitive neuroectodermal tumors, retinoblastomas, and rhabdomyosarcomas.

The step of measuring functional impacts of tumor-derived exosomes on immune cells to create a cancer-specific response profile or reference response profile can involve one or more mathematical steps or mathematical algorithms. Any suitable type or types of mathematical steps or mathematical algorithms can be used.

Preferably a plurality of different functional impact types are used to create a cancer-specific response profile or reference profile. For example, different functional impact types may correlate with different expression levels of a variety of markers on or in the immune cell.

In some embodiments measuring each type of functional impact of tumor-derived exosomes on immune cells to create a cancer-specific response profile (or reference profile) can comprise the step of quantifying the functional impact. For example, this can comprise quantifying the expression level of one or more different markers on or in the immune cell.

In some embodiments measuring the functional impact of tumor-derived exosomes on immune cells to create a cancer-specific response profile (or reference profile) can comprise the step of calculating a first ‘Parameter’ score based on the functional impact, normalized with respect to a control. The first Parameter score can be calculated by dividing the geometric mean for the functional impact by an average geometric mean for the control, and then log-2 transforming to obtain the first Parameter score for that functional impact.

The first Parameter score can be calculated in accordance with:

$$Parameter Score ( {M}_{i} ) = {log}_{2} \frac{{gMFI}_{i} of sample}{Average {gMFI}_{i} of control}$$

wherein gMFIᵢ is a geometric mean of the functional impact.

For example, this can comprise calculating a first Parameter score based on the expression level of a marker on the immune cell normalized with the expression level of the marker on a control immune cell.

The first Parameter score can be calculated in accordance with:

$$Parameter Score ( {M}_{i} ) = {log}_{2} \frac{{gMFI}_{i} of sample}{Average {gMFI}_{i} of control}$$

wherein gMFIᵢ is a geometric mean fluorescence intensity (gMFI) of the expression level of the marker on the immune cell.

In some embodiments measuring the functional impact of tumor-derived exosomes on immune cells to create a cancer-specific response profile (or reference profile) can comprise the step of calculating a second (‘Exo’) score based on a mean absolute value of the first Parameter score.

The second Exo score can be calculated in accordance with:

$$Exo Score = \frac{{\sum }_{i = 1}^{n} \vert {M}_{i} \vert}{n}$$

wherein the Mi is the first Parameter score and n is the number of Parameter scores.

In some embodiments measuring the functional impact of tumor-derived exosomes on immune cells to create a cancer-specific response profile (or reference profile) can comprise the step of calculating a third ‘Deviation’ score based on a mean of the absolute value of an average normalized deviation of the first Parameter score.

The third Deviation score can be calculated in accordance with:

$$Deviation Score = \frac{{Σ}_{i = 1}^{n} \vert \overline{{NPD}_{ι}} \vert}{n}$$

wherein the NPD comprises a normalized parameter deviation calculated in accordance with:

$$Normalized parameter deviation ( NPD ) = \frac{{x}_{i} - {M}_{i}}{\vert {M}_{i} \vert}$$

and wherein x is the Parameter score of a test sample for functional impact i and M is the identified Parameter score for that functional impact.

For example, where the first Parameter score is based on the expression level of a immune cell marker, the third score can comprise a deviation score calculated in accordance with

$$Deviation Score = \frac{{Σ}_{i = 1}^{n} \vert \overline{{NPD}_{ι}} \vert}{n}$$

wherein the NPD comprises a normalized parameter deviation calculated in accordance with:

$$Normalized parameter deviation ( NPD ) = \frac{{x}_{i} - {M}_{i}}{\vert {M}_{i} \vert}$$

and wherein x is the parameter score of a test sample for marker i and M is the identified parameter score for that marker.

Preferably, a third Deviation score less than 0.1 indicates matching to a cancer type in the database.

In some embodiments, the functional impact can be selected for inclusion in the response profile after conducting linear regressions and Spearman's rank-order correlation tests of first Parameter score data. In particular, the functional impact (eg. the expression marker) can be selected for inclusion of the response profile by conducting linear regressions and Spearman's rank-order correlation tests of first Parameter score against doses. Parameters can be selected if their correlation coefficient ρ and coefficient of determination R² fulfil one of the following conditions and pass visual checking:

Parameter selection:

|ρ|>0.3 and R²>0.2

|ρ|>0.4 and R²>0.1

|ρ|>0.2 and R²>0.3

In some embodiments the student t-test can be conducted and the magnitude of the differences between the mean of healthy and tumor groups can be calculated. Parameters can be selected if:

$$p < 0.05 or \vert \overline{{M}_{t u m o r}} - \overline{{M}_{H e a l t h y}} \vert > 0.2$$

In some embodiments the first Parameter score, the second Exo score and/or the third Deviation score can be used in creating cancer-specific response profiles.

In some embodiments the first Parameter score, the second Exo score and/or the third Deviation score can be used in creating reference profiles from subjects known to have cancer.

In some embodiments, the first Parameter score, the second Exo score and/or the third Deviation score can be used when comparing the cancer specific response profile of a subject to reference profiles.

In some embodiments the second Exo score can be used to give an overall ‘yes’ or ‘no’ answer as to whether cancer is present in a subject.

In some embodiments, the third Deviation score can be used to determine the type of cancer present in a subject in that it reflects the closeness of a response profile created for a subject to a reference response profile.

In some embodiments, the cancer specific response profile of the subject and reference profile can each be in the form of an immune response signature barcode.

In some embodiments, with regard to markers expressed on or in an immune cell (ie. representing a type of functional impact), the method can comprise:

(a) calculating a first Parameter score based on the expression level of the marker on the immune cell normalized with the expression level of the marker on a control immune cell;

(b) calculating a second Exo score based on a mean absolute value of the first Parameter score;

(c) calculating a third Deviation score based on a mean of the absolute value of an average normalized deviation of the first Parameter score; and

(d) comparing the first Parameter score, the second Exo score, and/or the third Deviation score of the subject response profile to a set of reference profiles prepared from subjects known to have cancer.

The test, assay, kit, apparatus or device for use or when used for detecting or diagnosing cancer or cancer type in a subject can comprise a reagent for culturing a tumor-derived exosome and an immune cell; and, a reagent for detecting the expression level of at least one marker on or in the immune cell.

The expression level of the marker can be measured using a detectable label. Any suitable label can be used for. For example, the label can be a detectable antibody.

The expression level of the marker can be measured using a device configured to detect and measure a detectable label.

The apparatus or device can be a flow cytometer/flow cytometry and/or real-time PCR (Polymerase Chain Reaction).

Preferably, the device is a flow cytometer.

The control immune cell can be an immune cell cultured with a non-diseased exosome or without exposure to exosome.

The non-diseased exosome can be a (healthy) non-diseased exosome.

The control can be an immune cell cultured without exosome, such as with buffer or media alone.

The exosome can be isolated from the extracellular fluid of the subject, such as blood.

The exosome can comprise tumor-derived exosome.

The test, assay, kit, apparatus or device can further comprise means for detecting the expression level of at least one marker on the immune cell.

The means for detecting the expression level of at least one marker on the immune cell can be a flow cytometer/flow cytometry.

The subject can be managed in any suitable way. As used herein, the term ‘managing’ (or ‘treating’) a subject or ‘management’ is such that the cancer is cured, healed, alleviated, relieved, altered, remedied, ameliorated, or improved. Management can include surgery and/or administering one or more therapeutic compounds in an amount effective to alleviate, relieve, alter, remedy, ameliorate, improve, or affect the illness or a symptom of the illness. Administration can include, but is not limited to, oral, sublingual, parenteral (e.g., intravenous, subcutaneous, intracutaneous, intramuscular, intraarticular, intraarterial, intrasynovial, intrasternal, intrathecal, intralesional or intracranial injection), transdermal, topical, buccal, rectal, vaginal, nasal, ophthalmic, via inhalation, and implants.

The method can comprise the step of isolating tumor-derived exosomes from a subject. The tumor-derived exosomes can be isolated from the subject in any suitable way. Preferably they are isolated by way of a liquid biopsy.

The method can comprise the step of culturing tumor-derived exosomes in the presence of immune cells, and this can be achieved in any suitable way. If culturing in the presence of T-cells, the presence of T-cell supporting molecules may be required.

The method can comprise the step of obtaining tumor-derived exosomes from the subject. This can comprise the step of culturing exosomes in the form of extracellular vesicles secreted by tumor cells of the subject in exosome-free culture medium. The extracellular vesicles/exosomes can have a size of about 20 nm to about 150 nm, or about 50 nm to about 140 nm, or about 80 nm to about 130 nm, or about 110 nm to about 120 nm, or 110+/−6 nm to 120+/−6 nm.

The method can comprise the step of testing tumor-derived exosomes for an exosomal marker, such as a marker typically associated with the exosome membrane. Any suitable type of marker can be tested. For example, a tetraspanin such as CD63 and/or CD9 can be tested.

The method can comprise the step of testing both the size of the exosome and the presence of an exosome marker for suitability for use in profiling functional impacts or creating a cancer-specific response profile.

The method can comprise the step of directly harvesting the tumor-derived exosomes from blood, without the need for a further exosome-purification step.

According to a fifteenth embodiment of the present invention, there is provided a method of measuring an expression level of a marker on an immune cell in contact with an exosome, wherein the method comprises: (a) culturing the exosome isolated from a subject in the presence of the immune cell; and (b) measuring the expression level of the marker on the immune cell.

According to a sixteenth embodiment of the present invention, there is provided a method of diagnosing a cancer in a subject in need thereof, wherein the method comprises:

(a) culturing an exosome isolated from the subject in the presence of an immune cell;

(b) measuring the expression level of a marker on the immune cell;

(c) calculating a first score based on the expression level of the marker on the immune cell normalized with the expression level of the marker on a control immune cell;

(d) calculating a second score based on a mean absolute value of the first score;

(e) calculating a third score based on a mean of the absolute value of an average normalized deviation of the first score; and

(f) comparing the first score, the second score, and the third score of the subject to a set of immune cell profile isolated from subjects known to have the cancer.

According to a seventeenth embodiment of the present invention, there is provided a method of quantifying the amount of an exosome in a subject, wherein the method comprises:

(a) culturing an exosome isolated from the subject in the presence of an immune cell;

(b) measuring the expression level of a marker on the immune cell;

(c) calculating a first score based on the expression level of the marker on the immune cell normalized with the expression level of the marker on a control immune cell; and,

(d) calculating a second score based on a mean absolute value of the first score, wherein the second score provides a quantitative assessment of the amount of exosome present.

According to an eighteenth embodiment of the present invention, there is provided an apparatus or device configured to perform the method of the fifteenth or sixteenth embodiment.

According to a nineteenth embodiment of the present invention, there is provided a kit comprising a reagent for culturing an exosome and an immune cell; and, a reagent for detecting the expression level of at least one marker on the immune cell.

The method of the fifteenth embodiment can further comprise: (c) calculating a first score based on the expression level of the marker on the immune cell normalized with the expression level of the marker on a control immune cell; (d) calculating a second score based on a mean absolute value of the first score; and, (e) calculating a third score based on a mean of the absolute value of an average normalized deviation of the first score, wherein the first score, the second score, and the third score are a set of immune cell profile against the cancer.

The method of the fifteenth embodiment can further comprise: one or more first parties performing the steps (a) and (b) and providing the expression level measurements of step (b) to a second party, the second party maintaining a database comprising the plurality of immune cell profiles selected for the plurality of cancer types; the second party performing steps (c), (d) and (e) for the expression level measurements; and the second party providing the set of immune cell profiles calculated from the expression level measurements and cancer cell types associated with the set of immune cell profiles determined from the database.

The method can further comprise the step of repeating steps (a) to (e) for a plurality of cancers to thereby have a plurality of immune cell profiles against the plurality of cancer types, the plurality of immune cell profiles selected for the plurality of cancer types being selected in accordance with predetermined criteria limitations.

The method can further comprise the step of generating an immune response signature barcode in response to the first score for the plurality of cancers to identify unique profiles of expression levels of markers on immune cells indicative of the plurality of cancer types.

Preferably, the predetermined criteria limitations include a mean of the first score differed by more than twenty percent between the immune cell expression and expression of a healthy control immune cell or the third score is less than five percent.

Parameter selection

|ρ|>0.3 and R²>0.2

|ρ|>0.4 and R²>0.1

|ρ|>0.2 and R²>0.3

Parameter selected if

$$p < 0.05 or \vert \overline{{M}_{t u m o r}} - \overline{{M}_{H e a l t h y}} \vert > 0.2$$

The expression level of the marker can be measured using a detectable label.

The expression level of the marker can be measured using a device configured to detect and measure a detectable label.

The device can be a flow cytometer/flow cytometry and/or real-time PCR (Polymerase Chain Reaction).

Preferably, the device is a flow cytometer.

The first score can comprise a parameter score calculated in accordance with:

$$Parameter Score ( {M}_{i} ) = {log}_{2} \frac{{gMFI}_{i} of sample}{Average {gMFI}_{i} of PBS or HEX controls}$$

Wherein gMFIᵢ is a geometric mean fluorescence intensity (gMFI) of the expression level of the marker on the immune cell.

The second score can comprise a score calculated in accordance with:

$$Exo Score = \frac{{Σ}_{i = 1}^{n} \vert {M}_{i} \vert}{n}$$

wherein the Mi is the first score and n is the number of parameter scores.

The third score can comprise a deviation score calculated in accordance with

$$Deviation Score = \frac{{Σ}_{i = 1}^{n} \vert \overline{{NPD}_{ι}} \vert}{n}$$

wherein the NPD comprises a normalised parameter deviation calculated in accordance with:

$$Normalized parameter deviation ( NPD ) = \frac{{x}_{i} - {M}_{i}}{\vert {M}_{i} \vert}$$

and wherein x is the parameter score of a test sample for marker i and M is the identified parameter score for that marker.

The control immune cell can be an immune cell cultured with a non-diseased exosome or without exposure to exosome.

The non-diseased exosome can be a (healthy) non-diseased exosome.

The control can be an immune cell cultured without exosome, such as with buffer or media alone.

The exosome can be isolated from the extracellular fluid of the subject, such as blood.

The exosome can comprise tumor-derived exosome.

The tumor-derived exosome can have a diameter of about 20 nm to about 150 nm, or about 50 nm to about 140 nm, or about 80 nm to about 130 nm, or about 110 nm to about 120 nm, or 110+/−6 nm to 120+/−6 nm.

The exosome can express exosomal membrane marker. The exosomal membrane marker can be CD63 or CD9.

The immune cell can be CD8 T cell, CD4 T cell, NK cell, or B cell. Preferably, the immune cell is CD8 T cell or CD4 T cell.

The marker can be selected from an immune cell activation marker, an immune cell proliferation marker, an immune cell exhaustion marker, an immune cell cytotoxicity marker, an immune cell cytotoxicity and apoptosis marker, or an immune cell inhibitory marker.

The activation marker can be CD69, CD25 or pSTAT5.

The proliferation marker can be ki67.

The exhaustion marker can be Tim3.

The cytotoxicity marker can be Granzyme B or IFNγ.

The cytotoxicity and apoptosis marker can be FasL.

The method inhibitory marker can be PD-1 or CTLA4.

The cancer can be renal carcinoma, colorectal carcinoma (colon cancer and/or rectal cancer), skin cancer (including basal cell carcinoma, cell carcinoma, squamous cell carcinoma and melanoma), leukemia, lymphoma, tumors of the central nervous system, breast cancer, prostate cancer, cervical cancer, uterine cancer, lung cancer, ovarian cancer, testicular cancer, thyroid cancer, astrocytoma, glioma, pancreatic cancer, mesotheliomas, gastric cancer, liver cancer, renal cancer including nephroblastoma, bladder cancer, oesophageal cancer, cancer of the larynx, cancer of the parotid, cancer of the biliary tract, endometrial cancer, adenocarcinomas, small cell carcinomas, neuroblastomas, adrenocortical carcinomas, epithelial carcinomas, desmoid tumors, desmoplastic small round cell tumors, endocrine tumors, Ewing sarcoma family tumors, germ cell tumors, hepatoblastomas, hepatocellular carcinomas, non-rhabdomyosarcome soft tissue sarcomas, osteosarcomas, peripheral primitive neuroectodermal tumors, retinoblastomas, and rhabdomyosarcomas.

The kit can further comprise means for detecting the expression level of at least one marker on the immune cell.

The means for detecting the expression level of at least one marker on the immune cell can be a flow cytometer/flow cytometry.

Context allowing, any feature or features described above can be used in connection with any one or more of the embodiments described above.

Context allowing, the feature or features of any one embodiment described above can be used in connection with any other embodiment described above.

### Description of Embodiments

Preferred features, embodiments and variations of the invention may be discerned from this section, which provides sufficient information for those skilled in the art to perform the invention. This section is not to be regarded as limiting the scope of any preceding section in any way.

### BRIEF DESCRIPTION OF FIGURES

Various embodiments of the invention will be described with reference to the following Figures.

FIG. 1: Characterizations and quantitative detection of TEXs produced in cancer cells culture. (A) Particle size of exosomes harvested from culture medium of different cancer cells was measured by Zeta View. Data represent the mean±standard deviation (SD) (n=10/group). (B) A sample histogram of particle size distribution of B16F10 TEXs. (C) Exosomes were linked to aldehyde/sulfate latex beads, followed by staining with anti-mouse CD63 and anti-mouse CD9. Flow cytometry analysis of fluorescence intensity of CD63 and CD9 on B16F10 TEXs coated beads and blank beads are shown. (D) Sample histogram of CD25 expression on activated CD4⁺ T-cells after treatment with 40×10⁸, 20×10⁸, 10×10⁸ and 0 EG7-OVA TEXs for two days. (E-G) T-cells were co-incubated with varying doses of TEXs from different cancer cells in the presence of supporting signals for 2 days, followed by markers staining and flow cytometry analysis. Parameter Score was calculated for each marker and Exo Score was computed with or without parameter selection. (E) Dose titration curves of Exo Scores for B16F10 TEXs and EG7 are presented with (solid lines) or without (dotted lines) parameter selection. (F) Dose titration curves of Exo Scores for A498 and HCT116 TEXs are shown after parameter selection. (G) Distinct patterns of Parameter Scores for B16F10, EG7-OVA, A498 and HCT116 TEXs. Pooled results are shown from at least three independent experiments for each cancer type. Act=activated.

FIG. 2: T-TEX diagnoses TEXs with interference from HEXs in blood. Blood obtained from C57Bl/6 mice was pooled before aliquoting. PBS or varying doses of TEXs from B16F10 and EG7-OVA cancer cells were spiked in to aliquots of blood. Spiked-in TEXs were re-harvested together with HEXs in the blood before co-culture with T-cells for 2 days. T-cell markers were stained and analyzed via flow cytometry. Parameter Score was calculated for each marker and Exo Score was computed after parameter selection. Data represent the mean±SD. Pooled results are shown from at least two independent experiments for each cancer type. (A) Dose titration curves of Exo Scores for B16F10 TEXs/HEXs mixture and EG7 TEXs/HEXs mixture. (B) Distinct patterns of Parameter Scores for B16F10 and EG7-OVA TEXs in the background of HEXs in blood.

FIG. 3: T-TEX diagnoses tumor-bearing mice against three types of tumor at the same time and identifies their cancer type. B16F10 melanoma cells (1×10⁶) were injected intravenously (i.v.) to induce lung metastases in C57Bl/6 mice for 10 days (n=14). EG7-OVA cells (1×10⁶) were injected s.c. into C57Bl/6 mice, and tumor was allowed to establish for 10 days (n=7). A498 renal carcinoma cells (4×10⁶) together with Matrigel® were inoculated s.c. into NCr nude mice for 10 weeks (n=27). Tumor-bearing mice and healthy control mice were then bled after the respective inoculation period, and exosomes in blood were harvested for T-TEX assay. (A) Exo Scores for healthy controls and mice with B16F10 lung metastasis after parameter selection. (B) Exo Scores for healthy mice and mice with EG7-OVA s.c. tumor after parameter selection. (C) Exo Scores for healthy mice and mice with A498 xenograft after parameter selection. (D) Distinct patterns of Parameter Scores for exosomes harvested from B16F10 lung metastasis, EG7-OVA s.c. tumor and A498 xenograft. (E) Exo Scores of A498 xenograft mice when diagnosed against B16F10 and EG7-OVA tumor pattern. (F) Normalized deviation of A498 xenograft mice from A498 Parameter Score pattern in each marker. (G) Normalized deviation of A498 xenograft mice from EG7-OVA Parameter Score pattern in each marker. N8=naïve CD8⁺ T-cells. N4=naïve CD4⁺ T-cells. A8=activated CD8⁺ T-cells. A4=activated CD4⁺ T-cells. (H) Deviation Scores of tumor-bearing mice when tested against B16F10, EG7-OVA and A498 tumor patterns. **, p<0.01; ***, p<0.001; ****, p<0.0001, by student t-test.

Herein the inventors describe, amongst other things, for the first time an approach to simultaneously diagnose multiple types of cancer by measuring/profiling functional impacts of their TEXs on T-cells, to create cancer-specific response profiles. The inventors have developed a diagnostic assay, T-TEX (named after the two key components in the assay), to capture the TEX-induced immune responses, designed algorithms to quantify the responses and have generated a cancer-specific data base of immune response profiles (reference cancer-specific profiles). The inventors have also created Exo Score to give an overall yes or no answer to cancer diagnosis, and Deviation Score to reflect the closeness of test samples to barcode patterns in the data base, thus scrutinizing the type of cancers. The inventors have detected, differentiated and quantified TEXs generated from four different cancer cell cultures. The inventors have also diagnosed tumor-bearing mice against three types of tumor at the same time with more than 89% sensitivity for each.

As T-TEX leverages on the functional impact of tumor signatures in the blood, it may circumvent the limitations in the current cancer biomarker development. It may also detect multiple types of cancer at the same time with a pre-built database, and serve as a first-line complimentary test to existing technology or standalone test to save potential patients/subjects from repetitive tests.

### Materials and Methods

### Materials

Heat inactivated fetal bovine serum (FBS) and Live/Dead fixable Aqua dead cell stain kit were obtained from Life Technologies (CA, USA). Concanavalin A Type VI (Con A) was obtained from Sigma-Aldrich (St. Louis, Mo.). Recombinant mouse interleukin-2 (IL-2) and interleukin-7 (IL-7) were obtained from eBioscience (MA, USA). Ficoll-Pague Plus was from GE Health Care (Waukesha, Wis.). Human peripheral blood mononuclear cells (PBMC), human interleukin-2 (IL-2), human interleukin-7 (IL-7), EasySep™ CD4⁺ or CD8⁺ T-cell Enrichment Kit for both mouse and human were bought from STEMCELL Technologies (Vancouver, Canada). Mouse and human anti-CD3/CD28 dynabeads and aldehyde/sulfate latex beads were purchased from Thermo Fisher Scientific (MA, USA). Matrigel® was obtained from BD Biosciences (CA, USA).

AccuCount rainbow fluorescent count beads (10.1 μm) were bought from Spherotech (Lake forest, IL). Anti-human ki67 Percp-Vio700 was from Miltenyi Biotec (BG, Germany). Anti-mouse CD16/32, anti-mouse CD8a APC, anti-mouse PD-1 APC-eFluor 780, anti-mouse Tim3 PE-Cy7, anti-mouse CD25-FITC anti-mouse GranzymeB-PE, anti-mouse CD4-eFluor 780, anti-mouse CTLA4 PE, anti-mouse FasL-Percp-eFluor 710, anti-mouse CD69 FITC, anti-mouse ki67 PE-Cy7, anti-mouse IFNγ, APC, anti-human CD4 APC-eFluor 780, anti-human CD8a APC-eFluor 780, anti-human CD69 APC, anti-human PD-1 PE-Cy7, anti-human CD25 FITC, anti-human Granzyme B PE, anti-human CTLA4 PE, anti-human Tim3 APC, anti-human IFNγ, FITC, anti-mouse CD16/32, human Fc Receptor binding inhibitor monoclonal antibody and Intracellular Fixation & Permeabilization Buffer Set were purchased from eBiosceince (San Diego, Calif.). All reagents were used as received unless otherwise noted.

### Animals and Cell Lines

The experimental protocol was approved by the Institutional Animal Care and Use Committee of Biological Resource Centre, Agency for Science, Technology and Research (A*STAR), Singapore. Six to eight week-old female C57Bl/6 mice and NCr nude mice were from the Singapore InVivos.

B16F10 mouse melanoma cells, EG7-OVA mouse lymphoma cells, A498 human renal carcinoma cells, HCT116 human colorectal carcinoma cells and S. aureus were acquired from American Type Culture Collection (Manassas, Va., USA).

### T-Cell Isolation and Activation

Spleens from C57Bl/6 mice were ground through a 70-μm cell strainer and red blood cells were removed by incubating with ACK lysis buffer (1 mL per spleen) for 3 min at 25° C. Naïve CD4⁺ or CD8⁺ T-cells were isolated from splenocytes directly via magnetic negative selection using an EasySep™ Mouse CD4⁺ or CD8⁺ T-cell Enrichment Kit, respectively. For activated CD8⁺ and CD4⁺ T-cells, splenocytes after ACK lysis were washed with ice cold PBS, and then cultured in T-cell medium with Con A at a final concentration of 2 μg/mL and murine IL-7 at 1 ng/mL at 37° C. for activation. After 2-day incubation, dead cells were removed by Ficoll-Pague Plus gradient separation, and CD8⁺ or CD4⁺ T-cells were isolated by EasySep™ Mouse CD8⁺ or CD4⁺ T-cell Enrichment Kit, respectively. Purified CD8⁺ or CD4⁺ T-cells were re-suspended at 0.75×10⁶/mL in T-cell medium containing 10 ng/mL recombinant murine IL-2. After 48 h, cells were washed in PBS and re-suspended in T-cell media for assays.

Human PBMCs were activated by Con A (2 μg/mL) and human IL-7 (1 ng/mL) at 37° C. for 2 days in T-cell medium. After removing dead cells by Ficoll-Pague Plus gradient separation, human CD8⁺ and CD4⁺ T-cells were isolated via EasySep™ human CD8⁺ or CD4⁺ T-cell Enrichment Kit, respectively. Purified CD8⁺ or CD4⁺ human T-cells were re-suspended at 1×10⁶/mL in T-cell medium containing 20 ng/mL of recombinant human IL-2. After 10 days, cells were washed in PBS and re-suspended in T-cell medium for assays.

Production of TEXs from cancer cell culture

FBS was spun at 110000 g for 3 hat 4° C. to remove exosomes. B16F10, A498 and HCT116 cancer cells were cultured in tumor medium (RPMI 1640 medium supplemented with 10% exosome-free FBS and 50 U/mL of Penicillin-Streptomycin), while EG7-OVA lymphoma cells were cultured in T-cells medium (tumor medium supplemented with Non-Essential Amino Acids, β-mercaptoethanol and pyruvate). After tumor cells grew confluent, tumor cell culture medium was harvested and spun down at 1000 g for 5 min at 4° C. Supernatant was collected and spun down at 10000×g for 30 min at 4° C. After the supernatant was collected and spun down by ultracentrifugation (Beckman Coulter, CA, USA) at 110,000 g for 70 min at 4° C., exosome pellets were re-suspended in 200 μI of PBS, quantified by Zeta View® (Particle Metrix GmbHAm, Meerbusch, Germany) and stored in −80° C. freezer.

### Generation and Harvest of TEXs in Blood

TEXs Spiked into Blood

Blood from 6 to 8 week-old healthy female C57Bl/6 mice was obtained via cardiac puncture. Different amounts of TEXs produced by B16F10 or EG7-OVA cells were spiked into the blood, and re-harvested together with HEXs via sequential centrifugations. The amount of TEXs in the mixture of TEXs and HEXs was assumed to be the same as those spiked into blood without loss. HEXs alone were also harvested from healthy mice blood without TEXs spiked in to serve as controls.

TEXs from Tumor-Bearing Mice

B16F10 melanoma cells were suspended at 1×10⁶ cells per 200 μL of PBS, and injected i.v. to induce lung metastases in C57Bl/6 mice for 10 days. For s.c. tumor models, EG7-OVA cells (1×10⁶) in 100 μL of PBS were injected s.c. into C57Bl/6 mice and tumor was allowed to establish for 10 days (100±45 cm²). In human tumor xenograft model, A498 renal carcinoma cells (4×10⁶) in 100 μL of PBS together with 100 μL Matrigel® were inoculated s.c. into NCr nude mice. After 10 weeks, tumor size was ˜114±67 cm². Tumor size was monitored before bleeding and tumor area was calculated as the product of 2 measured orthogonal diameters (D₁×D₂). Both healthy and tumor-bearing mice were bled (800-1000 μL) via cardiac puncture at respective time points to harvest HEXs and TEXs in the presence of background HEXs.

TEXs Harvest from Blood

Murine or human blood was spun at 3000 g for 5 min at 4° C. to obtain plasma that was further spun at 10000 g for 30 min at 4° C. Supernatant was then centrifuged at 110,000 g for 70 min at 4° C. Exosome pellets were re-suspended in 100 μI of PBS and stored in −80° C. freezer.

### Immune Response Assays

Murine naïve CD8⁺ T-cells (5×10⁴), naïve CD4⁺ T-cells (5×10⁴), activated CD8⁺ T-cells (5×10⁴) and activated CD4⁺ T-cells (5×10⁴) were each treated with PBS or an equivalent volume of varying doses of TEXs (in PBS) produced by B16F10 and EG7-OVA cancer cells in vitro. HEXs and TEXs/HEXs mixture harvested from the same volume of mouse blood were used in place of PBS and TEXs in PBS for assays to detect spiked-in TEXs, B16F10 lung metastasis, B16F10 and EG7 s.c. tumor. Naïve CD8⁺ and naïve CD4⁺ T-cells were supplemented with 1 μL of anti-mouse CD3/CD28 dynabeads while activated CD8⁺ and CD4⁺ T-cells were supplied with murine IL-2 with a final concentration of 8 ng/mL. Total volume per well was topped up to 120 μL with T-cell medium. T-cells were co-cultured with exosomes in the presence of supporting signals at 37° C. for 2 days before flow cytometry analysis.

For assays with exosomes from A498 and HCT116 cell lines, blood of A498 xenograft tumor-bearing mice and lung cancer patients, human T-cells, human IL-2 (16 ng/mL) and anti-human CD3/CD28 dynabeads were used while the rest of the setup remained the same.

### Flow Cytometry Analysis

After co-incubation with exosomes for 2 day, T-cells were added with counting beads, spun down and washed 2× with ice cold PBS before Aqua Live/Dead staining. T-cells were then washed 1× in FACS buffer and blocked by anti-mouse CD16/CD32 or anti-human FcR binding inhibitor monoclonal antibody before splitting into two halves for surface-staining of CD8, CD4, CD25, Tim3, CTLA4, PD-1, FasL, CD69 and pSTAT5. After washing 2× in FACS buffer, samples were fixed and permeabilized in eBioscience Intracellular Fixation & Permeabilization Buffer Set, followed by staining for ki67, Granzyme B and IFNγ. After intracellular staining, cells were washed 1× in FACS buffer and re-suspended in FACS buffer before analyzing on a BD LSR II or Celesta flow cytometer. All data were processed using FlowJo software.

### Data Analyses

Parameter Score

Flow cytometry data of every sample was processed to compute geometric Mean Fluorescence Intensity (gMFI) for each stained marker. All gMFI values were normalized to the average of PBS controls if TEXs were from in vitro cancer cell culturing or HEXs controls if TEXs were harvested from blood. Normalized gMFI value was then log-2 transformed to obtain parameter score (M) for that marker.

$$Parameter Score ( {M}_{i} ) = {log}_{2} \frac{{gMFI}_{i} of sample}{Average {gMFI}_{i} of PBS or HEX controls}$$

### Parameter Selection

For dose titration and spiked-in experiments, Spearman's rank-order correlation and linear regression were performed on dose and parameter score data. Parameters were selected if their correlation coefficient ρ and coefficient of determination R2 fulfilled one of the following conditions and passed visual checking:

1. |ρ|>0.3 and R²>0.2

2. |ρ|>0.4 and R²>0.1

3. |ρ|>0.2 and R²>0.3

For In assays for murine tumor models and human cancer patients, student t-test was conducted and the magnitude of the differences between the mean of healthy and tumor groups was calculated. Parameters were selected if

$$p < 0.05 or \vert \overline{{M}_{t u m o r}} - \overline{{M}_{H e a l t h y}} \vert > 0.2$$

### Exo Score

Exo Score was the mean absolute values of n parameter scores.

$$Exo Score = \frac{{Σ}_{i = 1}^{n} \vert {M}_{i} \vert}{n}$$

### Deviation Score

Normalized parameter deviation is defined as following where x is the parameter score of a test sample for marker i, while M is the identified parameter score for that marker.

$$Normalized parameter deviation ( NPD ) = \frac{{x}_{i} - {M}_{i}}{\vert {M}_{i} \vert}$$

Deviation Score is the mean of the absolute values of average NPD,

$$Deviation Score = \frac{{Σ}_{i = 1}^{n} \vert \overline{{NPD}_{ι}} \vert}{n}$$

FlowJo was used to compute all gMFI values. Data processing and statistical analyses were performed using RStudio (Version 1.0.153) and GraphPad Prism software. All values and error bars are mean±SD except where indicated differently.

### Results and Discussion

### Design of Diagnostic Assay T-TEX to Detect TEX-Induced Immune Responses

B16F10 mouse skin melanoma cells, A498 human renal carcinoma cells and HCT116 human colorectal carcinoma cells were cultured to generate representative TEXs from different tumor types and species. Since the inventors' diagnostic assay relied on the TEX-induced immune responses, EG7-OVA mouse lymphoma cells, a type of cancer cells originating from immune system itself was also included to evaluate whether T-TEX would also be applicable to immune system cancer.

For immune responses screening, the inventors used naïve CD8⁺ T-cells, naïve CD4⁺ T-cells, activated (Act) CD8⁺ T-cells or Act CD4⁺ T-cells to co-culture with TEXs in the presence of T-cell supporting molecules. For TEXs from B16F10 and EG7-OVA cells, T-cells from mouse spleens were used while for TEXs from A498 and HCT116 cells, T-cells from human peripheral blood mononuclear cells (PBMC) were employed. After 2 day of co-culture, various T-cell surface and intracellular markers were stained and analyzed via flow cytometry to provide insights about the TEXs. The markers screened include activation markers (CD69, CD25, pSTAT5), proliferation marker (ki67), exhaustion marker (Tim3), cytotoxicity marker (Granzyme B), protein crucial for cytotoxicity and immune cell apoptosis (FasL)³³ and those involved in immune checkpoint inhibitory signaling pathways (PD-1, CTLA4).

### T-TEX Detects Dose-Dependent Immune Responses to TEXs Generated in Cancer Cell Culture

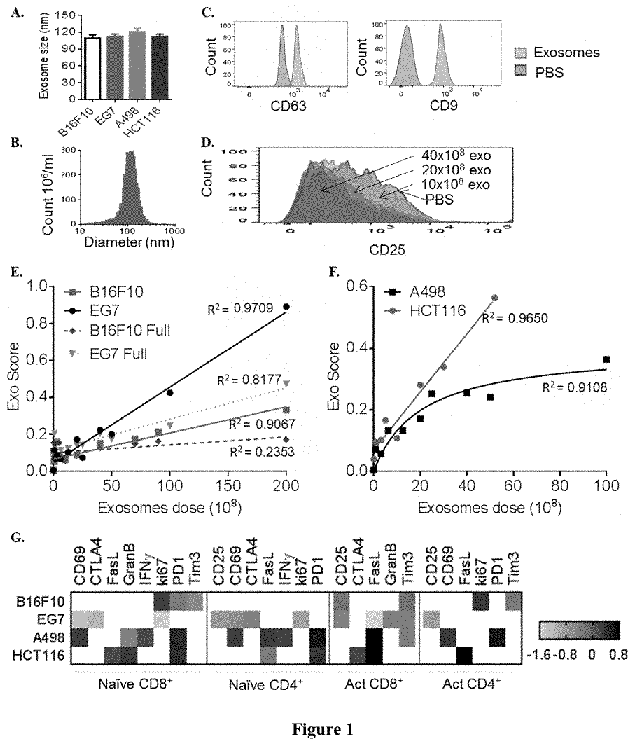

Extracellular vesicles (EVs) secreted by tumor cells cultured in exosome-free medium were harvested from culture medium via sequential centrifugations. The yielded vesicles had a mean size ranging from 110±6 nm to 120±6 nm for different types of cancer cells (FIG. 1A), falling into the size range for exosomes. A typical histogram of the size distribution of EVs from B16F10 is shown in FIG. 1B. In addition, harvested B16F10 EVs were tested positive for tetraspanins CD63 and CD9 (FIG. 10), which are exosome biomarkers associated with the exosomal membrane³⁴. These combined indicated that the EVs produced from cancer cell culture could be used as TEXs for the diagnostic assay.

At the end of T-TEX, the inventors obtained fluorescence intensity of markers in the designed panel as output. Sample histograms of fluorescence intensity of CD25 on T-cells after treatment with varying doses of TEXs were shown (FIG. 1D). CD25 expression was quantified by computing its geometric mean fluorescence intensity (gMFI), and normalized to the average gMFI of PBS controls so that CD25 expression could be compared fairly to other markers regardless of their default expression levels. The normalized CD25 expression was then log-2 transformed to give the Parameter Score of CD25. After computing Parameter Scores for all markers at different doses of TEXs, the inventors selected markers by conducting linear regressions and Spearman's rank-order correlation tests of Parameter Score against doses. For the diagnostic assay to be quantitative, markers demonstrating stronger linear dose-dependent responses will be favored (large R² value in linear regression). However, some of the marker responses might plateau after a certain dose, thus yielding a poorer linear fit. These parameters might still enhance the sensitivity of the assay at low concentration of TEXs, which would be useful for early stage cancer detection. These parameters can be recruited due to their high correlation coefficient in Spearman's rank-order test.

The inventors then calculated Exo Score, the mean of absolute values of Parameter Score for selected markers, to demonstrate the average magnitude of deviation per parameter of treated samples away from the controls. Without parameter selection, dose titration curve of Exo Score exhibited poor linear fits as R² was 0.2353 and 0.8117 for B16F10 and EG7-OVA TEXs, respectively (FIG. 1E dotted lines). Parameter selection significantly improved the R² value to 0.9067 and 0.9069 and increased the sensitivity of the assay by doubling the magnitude of change (steeper slope) (FIG. 1E). The inventors also managed to obtain unidirectional dose-dependent Exo Scores for TEXs from HCT116 (R²=0.9650 in linear fitting) and A498 cells (R²=0.9108 in Michaelis-Menten fitting) (FIG. 1F). Thus, not only could Exo Score detect the presence of TEXs generated from different types of cancer cells, it was also a quantitative assessment of the amount of TEXs present. Furthermore, the patterns of selected markers and their corresponding Parameter Scores were distinct among all four types of TEXs (FIG. 1G), demonstrating the possibility of using Parameter Score pattern to differentiate the types of cancer.

### T-TEX Identifies TEXs in the Background of Healthy Cell Derived Exosomes in Blood

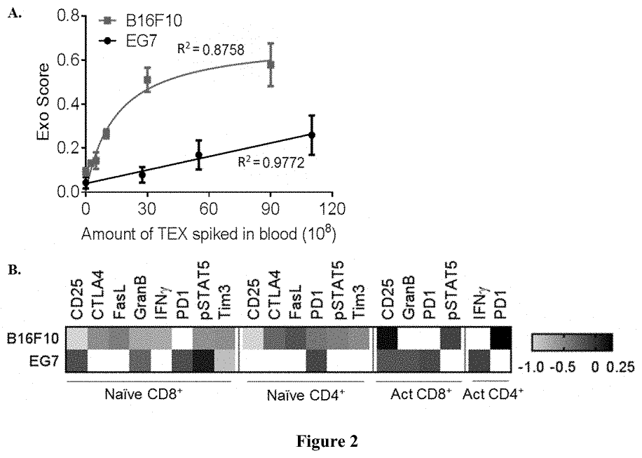

Exosomes secreted by healthy cells are present abundantly in blood^{22, 35, 36}, and they might affect the function of immune cells in T-TEX. To better mimic the real clinical setting, the inventors sought to evaluate whether Exo Score and Parameter Score could detect TEXs in the background of heathy cell derived exosomes (HEXs) from blood. Varying doses of B16F10 and EG7-OVA TEXs were spiked into healthy mice blood. The added TEXs were re-harvested together with HEXs originally in the blood via sequential centrifugation, and the mixture of TEXs and HEXs was tested by the inventors' assay. HEXs harvested from an equivalent volume of blood without TEX spiked in were used as controls to be normalized to. The Exo Score of EG7-OVA TEXs still exhibited a linear relationship with doses (R²=0.9772), while that of B16F10 TEXs was better fitted by Michaelis-Menten model (R²=0.8758) as Exo Score plateaued after 30×10⁸ dose (FIG. 2A). Due to the loss of exosomes during sequential centrifugation steps, the actual amount of TEXs used in the assays should be smaller than the indicated spiked-in amount, and the Exo Score curves might represent the responses in a lower range of doses. Nevertheless, Exo Score still detected TEXs with interference from HEXs in blood. It showed that T-TEX could diagnose cancer by using exosomes directly harvested from blood without the need to isolate TEXs. As expected, the patterns of selected markers and their corresponding Parameter Scores varied substantially from the results obtained in the last section (FIG. 1G, FIG. 2B) due to the interference of HEXs on T-cells in the assays. However, the patterns were still significantly different between B16F10 and EG7-OVA TEXs (FIG. 2B). Therefore, Parameter Scores could still be used to differentiate TEXs secreted by the two types of cancer cells.

T-TEX Diagnoses Tumor-Bearing Mice and Identifies their Respective Cancer Type

The inventors next evaluated T-TEX in the diagnosis of tumor-bearing mice. The inventors tested their assay in three tumor models, B16F10 murine lung metastasis model, EG7-OVA murine subcutaneous (s.c.) tumor model and A498 human tumor xenograft in immunodeficient mice to represent tumors from different origins, locations and species. Blood from healthy mice was used as controls.

As it was difficult to quantify the amount of TEXs in mice, the inventors changed their parameter selection criteria to the following: 1) mean Parameter Score differed more than 0.2 between healthy and tumor-bearing mice to improve sensitivity of the assay; 2) p-value in student t-test was smaller than 0.05 to increase the probability that the differences between healthy and tumor groups were not due to chance.

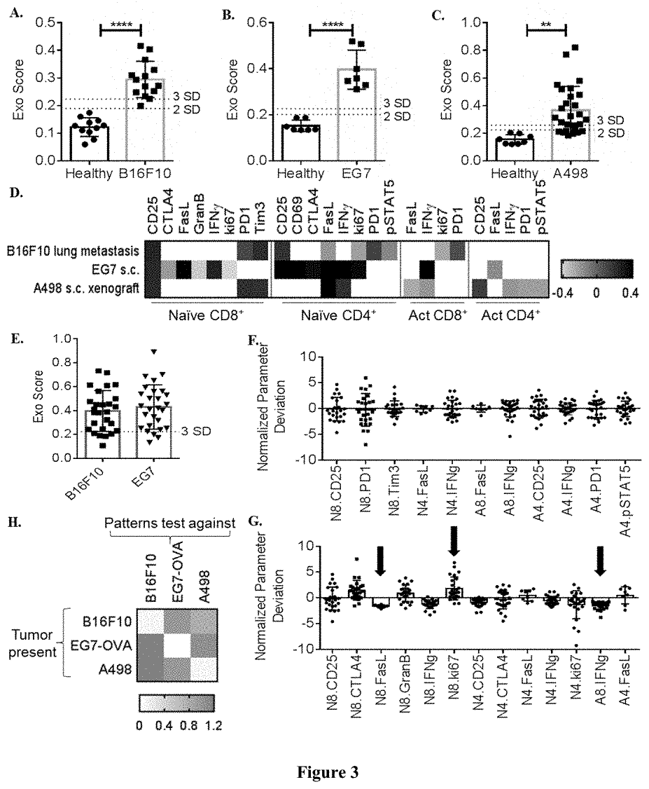

Compared to healthy mice, the Exo Scores of tumor-bearing mice were all significantly higher (FIG. 3A-C). The sensitivity of T-TEX was 93% and 100% for B6F10 and EG7-OVA tumor, respectively, with cut-off at 3 SDs above the mean of healthy controls. The sensitivity increased to 100% for both types of tumor with cut-off at 2 SDs above healthy control mean. The sensitivity of their assay to human cancer cell A498 in xenograft model was 93% (1 SD), 89% (2 SD), or 78% (3 SD) (FIG. 3C). The lower sensitivity in xenograft model might be due to the larger variation in tumor sizes by the time of bleeding. In addition, three tumor models all have their own distinctive patterns of eligible parameters and Parameter Scores (FIG. 3D).

Despite Exo Score was crucial in determining parameter patterns for different cancer type and could give an overall yes or no answer to diagnosis, it might not be able to differentiate types of cancers during the actual diagnosis stage. For example, when mice with A498 xenograft were diagnosed against B16F10 and EG7-OVA, more than 70% of mice were tested positive as their Exo Scores computed according to patterns for B16F10 and EG7-OVA were higher than the respective cut-off of 3 SDs (FIG. 3E). Thus, the inventors need another indicator to inform them about the specific type of cancer. A close look at the data revealed that the normalized parameter deviation of A498 tumor bearing mice from A498 pattern was random (FIG. 3F). On the other hand, test data of mice with A498 tumor exhibited strong directional changes in comparison to EG7-OVA pattern (FIG. 3G). Deviation Score, mean of the absolute values of average parameter deviation, was designed to capture the deviation of test samples from any known cancer patterns. Mice with A498 tumor showed Deviation Score larger than 1 to B16F10 and EG7 patterns, while only 0.1 to A498 pattern, indicating the tumors are A498 (FIG. 3H). Similarly, B16F10 and EG7-OVA tumor-bearing mice have high Deviation Scores when tested against other types of tumor, but not to the tumor they possessed (FIG. 3H). These results illustrated that Exo Score and Deviation Score could work together to identify the tumor-bearing mice, as well as specifying the type of cancer.

### CONCLUSIONS

The inventors have demonstrated a cancer diagnostic test, T-TEX, which can simultaneously detect multiple types of cancer by profiling functional impacts of their TEXs on T-cells. The inventors created Exo Score to give an overall yes or no answer to diagnosis, and Deviation Score to reflect the consistency of test samples to response patterns in the database, thus scrutinizing the type of cancer. T-TEX detects and quantifies TEXs from four different cancer cell lines and diagnoses mice against three types of tumor at the same time with more than 89% sensitivity for each. In the future, the assay can be expanded to use other types of immune cells such as Natural Killer (NK) cells and B cells for cancer.

Overall, as T-TEX leverages on the functional impacts instead of content of tumor signatures in blood, it will circumvent the limitations involved in current cancer biomarker development. With a pre-built database, it can also detect multiple types of cancer at the same time, thus serving as a first-line complimentary test to existing technology or a standalone test to minimize the burden of repeated testing.

### REFERENCES

1 Siravegna, G., Marsoni, S., Siena, S. & Bardelli, A. Integrating liquid biopsies into the management of cancer. Nat Rev Clin Oncol 14, 531-548 (2017).
2 Crowley, E., Di Nicolantonio, F., Loupakis, F. & Bardelli, A. Liquid biopsy: monitoring cancer-genetics in the blood. Nat Rev Clin Oncol 10, 472-484 (2013).
3 Karachaliou, N., Mayo-de-Las-Casas, C., Molina-Vila, M. A. & Rosell, R. Real-time liquid biopsies become a reality in cancer treatment. Ann Transl Med 3, 36 (2015).
4 Haber, D. A. & Velculescu, V. E. Blood-based analyses of cancer: circulating tumor cells and circulating tumor DNA. Cancer Discovery 4, 650-661 (2014).
5 Pantel, K. Blood-based analysis of circulating cell-free DNA and tumor cells for early cancer detection. PLoS Med 13, e1002205 (2016).
6 Ilie, M. et al. Current challenges for detection of circulating tumor cells and cell-free circulating nucleic acids, and their characterization in non-small cell lung carcinoma patients. What is the best blood substrate for personalized medicine? Ann Transl Med 2, 107 (2014).
7 Hong, B. & Zu, Y. Detecting circulating tumor cells: Current challenges and new trends. Theranostics 3, 377-394 (2013).
8 Simons, M. & Raposo, G. Exosomes—Vesicular carriers for intercellular communication. Curr Opin Cell Biol 21, 575-581 (2009).
9 El-Andaloussi, S., Mager, I., Breakefield, X. O. & Wood, M. J. Extracellular vesicles: Biology and emerging therapeutic opportunities. Nat Rev Drug Discov 12, 347-357 (2013).
10 Raposo, G. & Stoorvogel, W. Extracellular vesicles: Exosomes, microvesicles, and friends. J Cell Biol 200, 373-383 (2013).
11 Kahlert, C. et al. Identification of double stranded genomic DNA spanning all chromosomes with mutated KRAS and p53 DNA in the serum exosomes of patients with pancreatic cancer. J Biol Chem 289, 3869-3875 (2014).
12 Skog, J. et al. Glioblastoma microvesicles transport RNA and proteins that promote tumour growth and provide diagnostic biomarkers. Nat Cell Biol 10, 1470-1476 (2008).
13 Byron, S. A., Van Keuren-Jensen, K. R., Engelthaler, D. M., Carpten, J. D. & Craig, D. W. Translating RNA sequencing into clinical diagnostics: Opportunities and challenges. Nat Rev Genet 17, 257-271 (2016).
14 Taylor, D. D. & Gercel-Taylor, C. MicroRNA signatures of tumor-derived exosomes as diagnostic biomarkers of ovarian cancer. Gynecol Oncol 110, 13-21 (2008).
15 Yoshioka, Y. et al. Ultra-sensitive liquid biopsy of circulating extracellular vesicles using ExoScreen. Nat Commun 5, 3591 (2014).
16 Melo, S. A. et al. Glypican-1 identifies cancer exosomes and detects early pancreatic cancer. Nature 523, 177-182 (2015).
17 Cancer Genome Atlas Research Network, Weinstein, J. N. et al. The Cancer Genome Atlas Pan-Cancer analysis project. Nat Genet 45, 1113-1120 (2013).
18 Aravanis, A. M., Lee, M. & Klausner, R. D. Next-generation sequencing of circulating tumor DNA for early cancer detection. Cell 168, 571-574 (2017).
19 Meacham, C. E. & Morrison, S. J. Tumor heterogeneity and cancer cell plasticity. Nature 501, 328-337 (2013).
20 Wan, J. C. M. et al. Liquid biopsies come of age: Towards implementation of circulating tumour DNA. Nat Rev Cancer 17, 223-238 (2017).
21 Heitzer, E., Ulz, P. & Geigl, J. B. Circulating tumor DNA as a liquid biopsy for cancer. Clin Chem 61, 112-123 (2015).
22 Li, M. et al. Analysis of the RNA content of the exosomes derived from blood serum and urine and its potential as biomarkers. Philos Trans R Soc Lond B Biol Sci 369, 20130502 (2014).
23 Goossens, N., Nakagawa, S., Sun, X. & Hoshida, Y. Cancer biomarker discovery and validation. Transl Cancer Res 4, 256-269 (2015).
24 Munson, P. & Shukla, A. Exosomes: potential in cancer diagnosis and therapy. Medicines (Basel) 2, 310-327 (2015).
25 Kunigelis, K. E. & Graner, M. W. The dichotomy of tumor exosomes (TEX) in cancer immunity: Is it all in the conTEXt? Vaccines (Basel) 3, 1019-1051 (2015).
26 Robbins, P. D. & Morelli, A. E. Regulation of immune responses by extracellular vesicles. Nat Rev Immunol 14, 195-208 (2014).
27 Whiteside, T. L. Tumor-derived exosomes and their role in tumor-induced immune suppression. Vaccines 4, 35 (2016).
28 Valenti, R. et al. Tumor-released microvesicles as vehicles of immunosuppression. Cancer Res 67, 2912-2915 (2007).
29 Abusamra, A. J. et al. Tumor exosomes expressing Fas ligand mediate CD8⁺ T-cell apoptosis. Blood Cells Mol Dis 35, 169-173 (2005).
30 Clayton, A., Mitchell, J. P., Court, J., Mason, M. D. & Tabi, Z. Human tumor-derived exosomes selectively impair lymphocyte responses to interleukin-2. Cancer Res 67, 7458-7466 (2007).
31 Clayton, A., Al-Taei, S., Webber, J., Mason, M. D. & Tabi, Z. Cancer exosomes express CD39 and CD73, which suppress T cells through adenosine production. J Immunol 187, 676-683 (2011).
32 Muller, L., Mitsuhashi, M., Simms, P., Gooding, W. E. & Whiteside, T. L. Tumor-derived exosomes regulate expression of immune function-related genes in human T cell subsets. Sci Rep 6, 20254 (2016).
33 Nguyen, T. & Russell, J. The regulation of FasL expression during activation-induced cell death (AICD). Immunology 103, 426-434 (2001).
34 Andreu, Z. & Yanez-Mo, M. Tetraspanins in extracellular vesicle formation and function. Front Immunol 5, 442 (2014).
35 Vlassov, A. V., Magdaleno, S., Setterquist, R. & Conrad, R. Exosomes: Current knowledge of their composition, biological functions, and diagnostic and therapeutic potentials. Biochim Biophys Acta 1820, 940-948 (2012).
36 Caby, M. P., Lankar, D., Vincendeau-Scherrer, C., Raposo, G. & Bonnerot, C. Exosomal-like vesicles are present in human blood plasma. Intl Immunol 17, 879-887 (2005).

1.-46. (canceled)

47. A method of (i) detecting cancer or cancer type in at least one subject or (ii) simultaneously testing for, or distinguishing between, multiple types of cancer in at least one subject, said method comprising the step of using a cancer-specific response profile, created based on the measurement of functional impacts of tumor-derived exosomes on immune cells in vitro, to identify the cancer or the cancer type in the at least one subject, wherein said tumor-derived exosomes are isolated from the at least one subject.

48. The method of claim 47, comprising the step of screening a plurality of said at least one subject for a prevalence of cancer type or cancer types, said method comprising the step of using a cancer-specific response profile created for each said subject, created based on the measurement of functional impacts of tumor-derived exosomes on immune cells in vitro, to identify the cancer or the cancer types in each said subject, wherein said tumor-derived exosomes are isolated from the subjects.

49. A method of managing a subject with a cancer type, said method comprising the steps of:
(1) using a cancer-specific response profile, created based on the measurement of functional impacts of tumor-derived exosomes on immune cells in vitro, to identify the cancer type in the subject, wherein said tumor-derived exosomes are isolated from the subject; an
(2) managing the subject if the subject has been found to have the cancer type.

50. A method of identifying whether a subject having a cancer type is responding to management of that cancer type, said method comprising the steps of:
(1) using a cancer-specific response profile, created based on the measurement of functional impacts of tumor-derived exosomes on immune cells in vitro, to identify the cancer type in the subject, wherein said tumor-derived exosomes are isolated from the subject; and
(2) comparing the respective cancer-specific response profile created before and during and/or after management of the cancer type, wherein a change in the cancer-specific response profile identifies the subject as having responded to the management of the cancer type.

51. A method of generating a response profile specific for a cancer type, said method comprising the steps of:
(1) measuring functional impacts of tumor-derived exosomes on immune cells in vitro, wherein said tumor-derived exosomes are isolated from a subject having a specific cancer type; and
(2) creating a cancer-specific response profile based on the functional impact specific for the cancer type.

52. The method of claim 51, wherein said cancer-specific response profile is used in a method selected from the group consisting of:
(i) detecting cancer or cancer type in at least one subject or (ii) simultaneously testing for, or distinguishing between, multiple types of cancer in at least one subject;
(iii) screening a plurality of subjects for a prevalence of cancer type or cancer types;
(iv) managing a subject with a cancer type, said method comprising the steps of:
(1) using the cancer-specific response profile to identify the cancer type in the subject; and
(2) managing the subject if the subject has been found to have the cancer type; and
(v) identifying whether a subject having a cancer type is responding to management of that cancer type.

53. The method of claim 52, wherein the immune cells comprise one or more immune cells selected from the group consisting of T-cells, natural killer (NK cells), and B cells.

54. The method of claim 53, wherein the immune cells are T-cells.

55. The method of claim 52, wherein creating the cancer-specific response profile comprises measuring one or more of the following selected from the group consisting of:
suppression of the function of immune cells; impairment of immune cell responses to stimulants;
promotion of expansion of regulatory immune cells; induction of apoptosis of cytotoxic immune cells; and immunostimulation.

56. The method of claim 52, wherein creating the cancer-specific response profile comprises measuring immunosuppression due to one or more of the following immunoregulatory molecules selected from the group consisting of: IL-10, TGF-β, PD-1, PDL-1, TRAIL, FasL, CD39 and CD73.

57. The method of claim 52, wherein creating the cancer-specific response profile comprises measuring immunostimulatory effect due to one or more of the following molecules selected from the group consisting of: tumor antigens and heat shock proteins.

58. The method of claim 52, wherein creating the cancer-specific response profile comprises measuring at least one expression level of a marker on and/or in an immune cell.

59. The method of claim 58, wherein the marker is selected from the group consisting of: an immune cell activation marker; an immune cell proliferation marker; an immune cell exhaustion marker; an immune cell cytotoxicity marker; an immune cell cytotoxicity and apoptosis marker; and an immune cell inhibitory marker.

60. The method of claim 52, wherein the cancer is selected from the group consisting of renal carcinoma, colorectal carcinoma, skin cancer, leukemia, lymphoma, tumors of the central nervous system, breast cancer, prostate cancer, cervical cancer, uterine cancer, lung cancer, ovarian cancer, testicular cancer, thyroid cancer, astrocytoma, glioma, pancreatic cancer, mesotheliomas, gastric cancer, liver cancer, renal cancer including nephroblastoma, bladder cancer, oesophageal cancer, cancer of the larynx, cancer of the parotid, cancer of the biliary tract, endometrial cancer, adenocarcinomas, small cell carcinomas, neuroblastomas, adrenocortical carcinomas, epithelial carcinomas, desmoid tumors, desmoplastic small round cell tumors, endocrine tumors, Ewing sarcoma family tumors, germ cell tumors, hepatoblastomas, hepatocellular carcinomas, non-rhabdomyosarcome soft tissue sarcomas, osteosarcomas, peripheral primitive neuroectodermal tumors, retinoblastomas, and rhabdomyosarcomas.

61. The method of claim 52, comprising the step of comparing the created cancer-specific response profile of the subject with one or more previously created reference cancer-specific response profiles, wherein each said reference profile was created based on a subject diagnosed with a particular type of cancer.

62. The method of claim 52, comprising the step of comparing the created cancer-specific response profile of the subject with a prebuilt database of reference cancer-specific response profiles, wherein matching or near matching subject and reference profiles indicate the type of cancer that the subject has.

63. The method of claim 52, wherein a plurality of different functional impact types are used to create a cancer-specific response profile.

64. The method of claim 52, wherein the subject is a human.

65. The method of claim 52, wherein the tumor-derived exosomes are isolated from a liquid biopsy taken from the subject.

66. The method of claim 47, wherein the immune cells are T-cells.
